(19) United States
(12) Patent Application Publication
(10) Pub. No.: US 2023/0355922 A1
(43) Pub. Date: Nov. 9, 2023

(54) MICROCATHETER

(71) Applicant: BOSTON SCIENTIFIC MEDICAL DEVICE LIMITED, Galway (IE)

(72) Inventor: Eduardo Moriyama, Richmond (CA)

(21) Appl. No.: 18/347,865

(22) Filed: Jul. 6, 2023

Related U.S. Application Data
(63) Continuation of application No. PCT/IB2022/050142, filed on Jan. 10, 2022.
(60) Provisional application No. 63/136,350, filed on Jan. 12, 2021.

(51) Int. Cl. A61M 25/00; A61B 18/14

(52) U.S. Cl. CPC A61M 25/0021; A61B 18/1492; A61B 2018/00839

(57) ABSTRACT

A microcatheter configured to have a geometry that is movable along a tortuous anatomy of the patient; and be positionable, at least in part, proximate to biological tissue of a patient; and emit an information signal, related to the biological tissue, to a medical system so that the medical system, in use, receives the information signal from the microcatheter and processes, in use, the information signal received from the microcatheter.

### CROSS-REFERENCES TO RELATED APPLICATIONS

This application is a continuation of and claims the benefit of International Application Number PCT/IB2021/050142, entitled “MICROCATHETER,” and filed Jan. 10, 2022, which claims the benefit of U.S. Provisional Application No. 63/136,350, entitled “MICROCATHETER,” and filed Jan. 12, 2021, which are hereby incorporated by reference in their entireties.

### TECHNICAL FIELD

This document relates to the technical field of (and is not limited to) catheters, and more specifically to microcatheters (and method therefor).

### BACKGROUND

Known medical devices are configured to facilitate a medical procedure, and help healthcare providers diagnose and/or treat medical conditions of sick patients.

### SUMMARY

It will be appreciated that there exists a need to mitigate (at least in part) at least one problem associated with existing (known) catheters. After much study of, and experimentation with, existing (known) catheters, an understanding (at least in part) of the problem and its solution have been identified (at least in part) and are articulated (at least in part) as follows:

Known catheters cannot be used to improve better outcomes for a given medical procedure.

What may be needed (to solve the problem) is a microcatheter configured to: have a geometry that is movable along a tortuous anatomy of the patient; and be positionable, at least in part, proximate to the biological tissue; and to emit an information signal, related to the biological tissue, to a medical system so that the medical system, in use, receives the information signal from the microcatheter and processes, in use, the information signal received from the microcatheter.

To mitigate, at least in part, at least one problem associated with the existing technology, there is provided (in accordance with a major aspect) an apparatus. The apparatus is usable with a medical system and with biological tissue of a patient. The apparatus includes and is not limited to (comprises) a microcatheter configured to: have a geometry being movable along a tortuous anatomy of the patient; and be positionable, at least in part, proximate to the biological tissue; and to emit an information signal, related to the biological tissue, to a medical system so that the medical system, in use, receives the information signal from the microcatheter and processes, in use, the information signal received from the microcatheter. A technical advantage associated with the microcatheter is improved (enhanced) quality of the information signal to be provided to the medical system on the basis that the microcatheter may be positioned relatively closer to the biological tissue so that the surgeon may be better able to arrive at a better outcome for a given medical procedure. There is an advantage for having the microcatheter is configured to: be sized to reach a position located relatively close to the biological tissue; and/or navigate tortuous anatomy of the patient, etc.

To mitigate, at least in part, at least one problem associated with the existing technology, there is provided (in accordance with a major aspect) a method. The method is usable with a medical system and biological tissue of a patient. The method includes and is not limited to using a microcatheter configured to: have a geometry being movable along a tortuous anatomy of the patient; and be positionable, at least in part, proximate to the biological tissue; and to emit an information signal, related to the biological tissue, to the medical system so that the medical system, in use, receives the information signal from the microcatheter and processes, in use, the information signal received from the microcatheter.

Other aspects are identified in the claims. Other aspects and features of the non-limiting embodiments may now become apparent to those skilled in the art upon review of the following detailed description of the non-limiting embodiments with the accompanying drawings. This Summary is provided to introduce concepts in simplified form that are further described below in the Detailed Description. This Summary is not intended to identify potentially key features or possible essential features of the disclosed subject matter and is not intended to describe each disclosed embodiment or every implementation of the disclosed subject matter. Many other novel advantages, features, and relationships will become apparent as this description proceeds. The figures and the description that follow more particularly exemplify illustrative embodiments.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 1 depicts a side view of an embodiment of a medical image (generated by a medical-imaging system) of a microcatheter positioned (at least in part) in a heart; and

FIG. 2 depicts a perspective view of an embodiment of the microcatheter of FIG. 1; and

FIG. 3, FIG. 4, FIG. 5, FIG. 6 and FIG. 7 depict side views of embodiments of the microcatheter of FIG. 1; and

FIG. 8, FIG. 9, FIG. 10, FIG. 11, FIG. 12, FIG. 13 and FIG. 14 depict side views of embodiments of the microcatheter of FIG. 1.

The drawings are not necessarily to scale and may be illustrated by phantom lines, diagrammatic representations and fragmentary views. In certain instances, details unnecessary for an understanding of the embodiments (and/or details that render other details difficult to perceive) may have been omitted. Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, and well-understood, elements that are useful in commercially feasible embodiments are often not depicted to provide a less obstructed view of the embodiments of the present disclosure.

### DETAILED DESCRIPTION OF THE NON-LIMITING EMBODIMENT(S)

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the disclosure is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the disclosure is limited to the subject matter provided by the claims, and that the disclosure is not limited to the particular aspects depicted and described. It will be appreciated that the scope of the meaning of a device configured to be coupled to an item (that is, to be connected to, to interact with the item, etc.) is to be interpreted as the device being configured to be coupled to the item, either directly or indirectly. Therefore, “configured to” may include the meaning “either directly or indirectly” unless specifically stated otherwise.

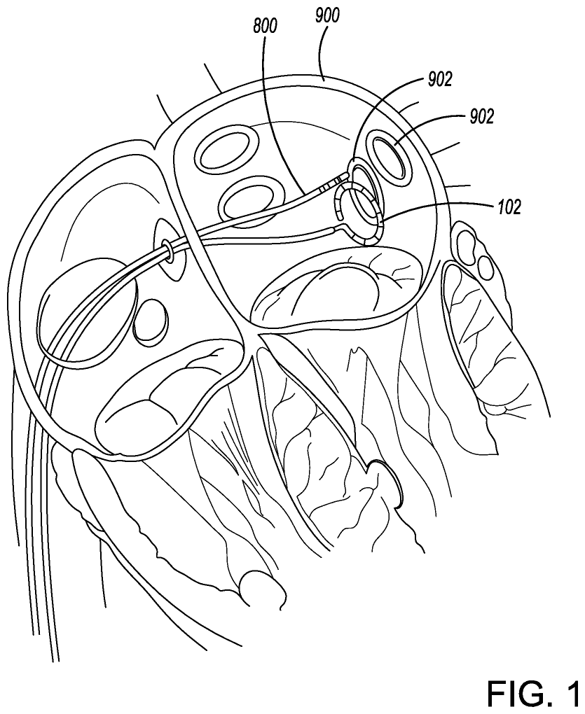

FIG. 1 depicts a side view of an embodiment of a medical image (generated by a medical-imaging system) of a microcatheter 102 positioned (at least in part) in a heart 900.

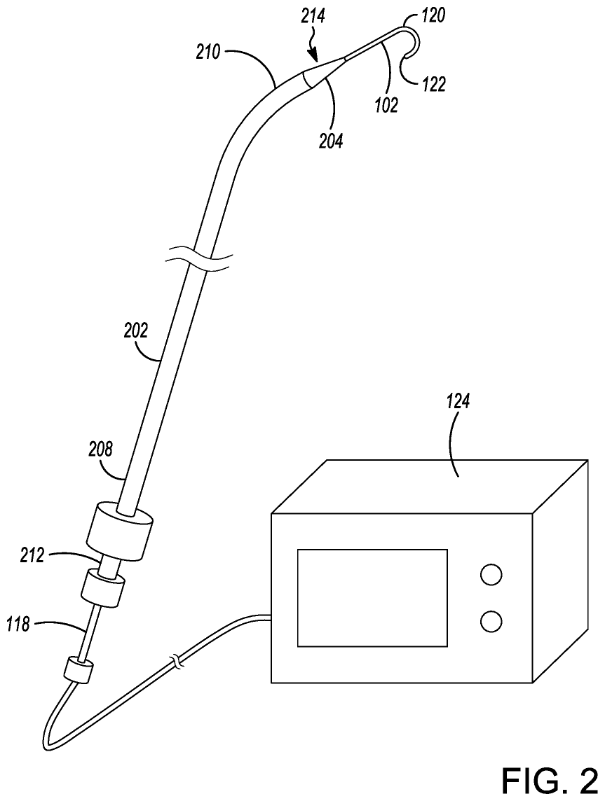

Referring to the embodiment as depicted in FIG. 1, the microcatheter 102 is configured to (applicable to a first major embodiment and to all other embodiments): (A) have a geometry being movable, at least in part, along a tortuous anatomy of the patient; and/or (B) be positionable, at least in part, (by the surgeon) proximate to biological tissue (such as, the interior biological tissue of the heart 900, etc.); and/or (C) to emit an information signal, related to the biological tissue, to a medical system (such as the medical system 124 depicted in FIG. 2, known and not depicted in FIG. 1) for the purpose of having the medical system 124, in use, receive, either directly or indirectly, and/or via wire or wirelessly, the information signal from the microcatheter 102. The microcatheter 102 is configured to be inserted into a confined space defined by a living body (the patient). Emission of the information signal, related to the biological tissue, may include having the microcatheter 102 configured to be selectively signal connectable, as or when required, to the medical system 124 (for the purpose of having the medical system 124 receive the information signal from the microcatheter 102 so that the medical system 124 may process the information signal that was received from the microcatheter 102).

Referring to the embodiment as depicted in FIG. 1, a technical advantage associated with the microcatheter 102 (applicable to the first major embodiment and to all other embodiments) is improved (enhanced) quality of the information signal to be provided to the medical system 124 (such as a medical-imaging system) on the basis that the microcatheter 102 may be positioned relatively closer to the biological tissue (that may receive medical treatment) so that the surgeon may be better able to arrive at a better outcome for a given medical procedure. There is an advantage for having the microcatheter 102 is configured to: (A) be sized to reach a position located relatively close to the biological tissue; and/or (B) navigate tortuous anatomy of the patient, etc.

Referring to the embodiment as depicted in FIG. 1, the microcatheter 102 includes (preferably) biocompatible material properties suitable for specific performance (such as, dielectric strength, thermal, electrical insulation, corrosion, water resistance, heat resistance, etc.), for compliance with industrial and/or regulatory safety standards (or compatible for medical usage), etc. Reference is made to the following publication for consideration in the selection of a suitable material: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew, [2014].

Referring to the embodiment as depicted in FIG. 1, the microcatheter 102 may be further configured to: (A) detect presence of the biological tissue (such as of the heart 900) that is positioned or located, at least in part, proximate to the microcatheter 102 (this is done, for instance or preferably, after the microcatheter 102, in use, is selectively connected to the medical-imaging system); and/or (B) transmit (at least one or more) information signals (indicating detection of the presence of the biological tissue) to the medical-imaging system (the systems and/or methods for transmitting the information signals from the microcatheter 102 to the medical-imaging system are well known, and are therefore not further described); and the medical-imaging system is configured to generate (form) a medical image (based on computations performed on the information signal that was provided by the microcatheter 102; said medical image is depicted in FIG. 1, by way of example). For this specific case, a technical advantage associated with the microcatheter 102 is improved (enhanced) quality of the medical image to be formed by the medical-imaging system on the basis that the microcatheter 102 may be positioned relatively closer to the biological tissue (that may receive medical treatment). There is an advantage for having the microcatheter 102 is configured to: (A) be sized to reach a position located relatively close to the biological tissue; and/or (B) navigate tortuous anatomy of the patient, etc.

Referring to the embodiment as depicted in FIG. 1, the microcatheter 102 may include a shape-memory material configured to be manipulated and/or deformed followed by a return to the original shape that the shape-memory material was set in (prior to manipulation). Shape-memory materials (SMMs) are known and not further described in detail. Shape-memory materials are configured to recover their original shape from a significant and seemingly plastic deformation in response to a particular stimulus being applied to the shape-memory material. This is known as the shape memory effect (SME). Superelasticity (in alloys) may be observed once the shape-memory material is deformed under the presence (an application) of a stimulus force.

Referring to the embodiment as depicted in FIG. 1, the medical system 124 may include, for instance (and not limited thereto) a medical-imaging system and any equivalent and/or similar system, such as (and not limited to) an EAMS (Electro Anatomical Measurement System), which is known and, therefore, not depicted (other types of medical systems are described or identified in this document). The medical image (generated by the medical-imaging system) is to be depicted in (on) a display device (known and not depicted) of the medical-imaging system, so that the surgeon may advantageously refer to the medical image during a medical procedure (thereby, advantageously improving the successful outcome for the patient). The EAMS (Electro Anatomical Measurement System (mapping system) may include fluoroscopy mapping systems (if so desired, but may not be preferred for some embodiments). The electroanatomical mapping system includes, preferably, a nonfluoroscopy mapping system, such as, and not limited to, (A) the CARTO EP (TRADEMARK) mapping system (manufactured by BIOSENSE WEBSTER based in the USA), (B) the ENSITE PRECISION (TRADEMARK) cardiac mapping system (manufactured by Abbott Laboratories based in the USA), (C) the LOCALISA (TRADEMARK) intracardiac mapping system (manufactured by MEDTRONICS INC., based in the USA), and (D) the RHYTHMIA HDx (TRADEMARK) mapping system (manufactured by Boston Scientific Corp., based in the USA).

Referring to the embodiment as depicted in FIG. 1, the medical-imaging system may be utilized to identify a portion of biological tissue that is to receive medical treatment (such as, the site of puncture, etc.). The portion of biological tissue may be determined through any suitable visualization method, such as (and not limited to): (A) fluoroscopy through the use of RO markers and/or distal electrode; and (B) angiography (simultaneous angiography or near simultaneous angiography) to determine the orientation and position of the distal end of the microcatheter; and (C) electro-anatomical mapping for real-time placement of the microcatheter and sheath with targets predetermined on CT or in real-time; and (D) echogenic markers or features on either the microcatheter or the supporting catheter which may enable use of ICE (Intracardiac Echocardiography) or TEE (Transesophageal Echocardiography) for delineation of etiology and optimal target site to avoid damaging surrounding vasculature, etc.

Referring to the embodiment as depicted in FIG. 1, in accordance with the first major embodiment, an elongated energy-emitting device 800 includes an energy emitter configured to be positioned in the interior of the heart 900 of the patient. An example of the elongated energy-emitting device 800 includes the BAYLIS (TRADEMARK) model POWER WIRE (TRADEMARK) RF guidewire, manufactured by BAYLIS MEDICAL COMPANY based in Canada. The energy-emitting device 800 is configured to: (A) be selectively connected to an energy source (known and not depicted, such as a radio-frequency source); and/or (B) be moved and positioned (by the surgeon) proximate to the biological tissue (preferably, the biological tissue is located in the interior of the heart 900 of the patient, etc.); and/or (C) selectively emit energy (such as, radio-frequency energy) from the energy source toward the biological tissue (of the heart 900) that is positioned proximate to the energy emitter (of the energy-emitting device 800) after the energy-emitting device 800, in use, is selectively connected to the energy source; this may be done in such a way that the energy (which is emitted toward the biological tissue) is used (at least in part) to form at least one instances of an ablated tissue portion 902 on the biological tissue (such as, of the heart 900). It will be appreciated that, for the purpose of assisting the surgeon in positioning the energy emitter of the energy-emitting device 800, the surgeon performing the medical procedure (for the formation of the ablated tissue portion 902) may, advantageously, utilize the medical image (as depicted in FIG. 1) that was generated by the medical-imaging system based on the information signal provided by the microcatheter 102. This arrangement may improve patient outcome. If so desired, the microcatheter 102 may transmit the information signals while the energy emitter (of the elongated energy-emitting device 800) emits energy.

Referring to the embodiment as depicted in FIG. 1, in accordance with a second major embodiment, the energy-emitting device 800 is not used (per se), and therefore for this case the microcatheter 102 is further configured to selectively emit (at least in part) energy (such as, radio-frequency energy) toward the biological tissue (of the heart 900) for the purpose of treating (such as, puncturing, ablating, etc.) the biological tissue; this is done (preferably) in such a way that the energy is used to form at least one instance of an ablated tissue portion 902 on the biological tissue (that is, after or while the medical image has been formed by the medical-imaging system). For this case, the microcatheter 102 is configured to: (A) be selectively connectable, as or when required, to an energy source (known and not depicted); and (B) selectively emit (in use) a signal to the medical system 124 (as depicted in FIG. 2) for the purpose of having the medical system receive a signal from the microcatheter 102; and (C) be selectively signal connectable, as or when required, to the medical system 124 for the purpose of having the medical system receive the information signal from the microcatheter 102 so that the medical system may process the information signal received from the microcatheter 102.

Referring to the embodiment as depicted in FIG. 1, in further accordance with the second major embodiment, the microcatheter 102 may be further configured (more preferably to: (A) detect (sense, respond to) the presence of biological tissue (such as, the heart 900) in response to the surgeon moving and positioning the microcatheter 102 proximate to (relative to) the biological tissue (this is done, preferably, after the microcatheter 102, in use, is selectively signal connected to the medical-imaging system); and/or (B) transmit (at least one or more) information signals associated with the biological tissue (that was detected by the microcatheter 102) to the medical-imaging system (this is done, preferably, in such a way that the information signals are used, preferably by the medical-imaging system, to form the medical image); and/or (C) assist the surgeon for the positioning (movement) of the microcatheter 102 (at a desired location in, or a desired position on, the medical image that was formed by the medical-imaging system); and/or (D) selectively emit energy (such as, radio-frequency energy) toward the biological tissue (of the heart 900) based on the medical image that was generated from the information signal provided by the microcatheter 102 (after the microcatheter 102, in use, is selectively connected to the energy source).

Referring to the embodiment as depicted in FIG. 1, a technical advantage may be associated with the microcatheter 102 (in accordance with the second major embodiment), which is improved (enhanced) quality of the medical image (similar to the advantage associated with the first major embodiment). Another a technical advantage may be associated with the microcatheter 102 (in accordance with the second major embodiment), which is improved streamlined workflow that may reduce the number of devices and/or device exchanges, thereby optimizing workflow pursuant to reducing complications during a medical procedure (such as, and not limited to, the PVI (Pulmonary Vein Isolation) procedure, thrombus formation, procedure time, and/or x-ray exposure to patients, etc.).

Referring to the embodiment as depicted in FIG. 1, in further accordance with the second major embodiment, the microcatheter 102 may be operated under different operational modes (as desired in any combination and/or permutation), such as: (A) the microcatheter 102 may be configured to not emit energy while the microcatheter 102, in use, detects the biological tissue (such as, of the heart 900) that is positioned proximate to the microcatheter 102; and/or (B) the microcatheter 102 may be configured to not emit energy (such as radio-frequency energy, etc.) while the microcatheter 102, in use, transmits (at least one or more of) the information signals that are associated with the biological tissue (that was detected by the microcatheter 102); and/or (C) the microcatheter 102 may be configured to not emit energy while the microcatheter 102, in use, assists the surgeon in positioning the microcatheter 102 (at a desired position or location of the medical image which was formed by the medical-imaging system); and/or (D) the microcatheter 102 may be configured to not detect biological tissue while the microcatheter 102, in use, selectively emits energy (such as, radio-frequency energy) toward the biological tissue (as indicated in, or formed by, the medical image that was generated from the information signal provided by the microcatheter 102).

Referring to the embodiment as depicted in FIG. 1, the microcatheter 102 may be utilized for assisting the surgeon to perform pulmonary vein isolation (PVI), which is a treatment for a kind (or type) of irregular heartbeat (arrhythmia) known as atrial fibrillation (also called AF or A-Fib). Pulmonary vein isolation is a type of cardiac ablation. Cardiac ablation works by scarring or destroying biological tissue located in the interior of the heart that triggers an abnormal heart rhythm. The microcatheter 102 may, if desired, include multiple electrodes configured to allow a single device to perform multiple tasks (such as for ablation, such as atrial fibrillation, etc.).

Referring to the embodiment as depicted in FIG. 1, the microcatheter 102 is, preferably, a small diameter catheter that is used in minimally invasive medical procedures, and is configured to deliver at least one or more devices. The microcatheter 102 is, preferably, small enough for navigating complex vasculature within the human body. For instance, the microcatheter 102 may have a diameter of about 0.70 to about 1.30 millimeters (mm). For instance, the microcatheter 102 may be used for guidewire support, device exchanges (where medical devices may be exchanged, etc.), to access distal anatomy, cross through a lesion, delivery of therapeutic embolism, injection of a contrast media, and/or perform other procedures, such as complex endovascular procedures. For instance, the microcatheter 102 may be steerable. For instance, the microcatheter 102 may be used in cardiac applications such as balloon delivery to improve vessel flow in elderly patients. For instance, the microcatheter 102 may be used to place and exchange guidewires and other interventional devices for diagnostic and therapeutic applications. For instance, the microcatheter 102 may include a lubricious coating. For instance, the microcatheter 102 may include an integrated steerable tip. For instance, the microcatheter 102 may include an angled tip for relatively easier penetration and deliverability. For instance, the microcatheter 102 may include a hydrophilic coating that may enhance navigation through tortuous vasculatures while a coil pitch increases flexibility and proximal pushability. For instance, the microcatheter 102 may be used in percutaneous coronary intervention.

Referring to the embodiment as depicted in FIG. 1, the microcatheter 102 is (preferably) configured to provide any one or more of the following configurations or functions (in any combination and/or permutation): (A) perform transseptal puncture; and/or (B) secure LA (Left Anterior Descending Artery of the heart) access; and/or (C) serve as an anchor; and/or (D) detect and collect a cardiac signal (diagnostic information); and/or (E) perform cardiac pacing; and/or (F) record EKG signals; and/or (G) support delivery of a therapy device; and/or (H) perform therapy (such as, treatment for Premature Ventricular Contractions); and/or (I) reduce or remove a need for exchanging access devices for multiple devices; and/or (J) function in each step of the PVI (pulmonary vein isolation) workflow, etc.

FIG. 2 (SHEET 2 of 7 SHEETS) depicts a perspective view of an embodiment of the microcatheter 102 of FIG. 1.

Referring to the embodiment as depicted in FIG. 2, the microcatheter 102 is (preferably) configured to: (A) be usable with a sheath 202; and (B) be usable with a dilator 204 configured to be received (at least in part) into the sheath 202; and (C) be received (at least in part) into the dilator 204. The sheath 202 and the dilator 204 may (then) be advanced over the microcatheter 102, to the desired location (such as, at the heart via the femoral vein, etc.). The microcatheter 102 may be directed to a desired site by the sheath 202 and the dilator 204. For instance, the microcatheter 102 may be inserted into the femoral vein (of the heart 900 of FIG. 1) and the distal tip of the microcatheter 102 may be placed in the desired location in the heart 900, such as the right artery, in the superior vena cava, etc.

Referring to the embodiment as depicted in FIG. 2, the sheath 202 is (preferably) configured to be usable for guiding other medical devices (such as the dilator 204, a therapeutic device such as a stent or shunt, etc.) toward a target location in a patient's body (such as, the superior vena cava (SVC) of the heart, etc.). The sheath 202 has a proximal sheath portion 208 and a distal sheath portion 210. The sheath 202 forms (has) a sheath lumen (not shown) extending along the elongated length of the sheath 202 from the proximal sheath portion 208 to the distal sheath portion 210. The sheath 202 may (optionally) have a fixed curve. The sheath 202 may (optionally) be configured to be steerable (that is, the curve of the sheath 202 may be changed, optionally, in more than one plane, as might be required).

Referring to the embodiment as depicted in FIG. 2, the sheath 202 may have a fixed curve. The sheath 202 may be configured to be steerable, and the microcatheter 102 may be steerable. The sheath 202 may have a small-bore steerable kit (such as, less than about 10 French diameter). The sheath 202 may have a large-bore Steerable (such as, greater than 10 French).

Referring to the embodiment as depicted in FIG. 2, the dilator 204 is configured to dilate a perforation (such as, a hole extending through a biological wall, etc.). The dilator 204 has a proximal dilator portion 212 and a distal dilator portion 214 having a dilating tip portion. The dilator 204 forms (has) a dilator lumen (not shown) that extends along an elongated length of the dilator 204 from the proximal dilator portion 212 to the distal dilator portion 214. The dilator 204 may be configured to have a fixed curve. The dilator 204 may be configured to be steerable (that is, the curve may be changed, optionally in more than one plane, etc.). The dilator 204 may be configured to be flexible to allow it to be compatible with a steerable sheath, etc.

Referring to the embodiment as depicted in FIG. 2, the dilator 204 is configured to (preferably, in any combination and/or permutation thereof): (A) be steerable; and/or (B) have an atraumatic distal tip; and/or (C) be bi-directional; and/or (D) have multi-plane steerability; and/or (E) have one or more open lumens; and/or (F) have the ability to curve; and/or (G) to provide direct placement of the distal microcatheter portion 120 for site-selection; and/or (H) have one or more visualization markers.

Referring to the embodiment as depicted in FIG. 2, the microcatheter 102 is (preferably) configured to provide any one or more of the following configurations (in any combination and/or permutation): (A) be steerable; and/or (B) change shape (such as a pulling system).

Referring to the embodiment as depicted in FIG. 2, the microcatheter 102 is (preferably) configured to provide any one or more of the following configurations (in any combination and/or permutation): (A) have any diameters, such as a diameter of about two French (2F); and/or (B) have a shaft made of polymer or other isolating material; and/or (C) have any suitable length, such as 180 centimeters (cm); and/or (D) has connectors (electrical connectors) configured for interfacing with different systems (such as, an energy source, a medical-imaging system, an ECG system, etc.); and/or (E) have a J-tip; and/or (F) be straight; and/or (G) be curved, circular, semi-circular, etc. (the diameter of the circle or curve may be optimum for a given medical procedure); and/or (H) have one or more sensors or electrodes located in the distal end of the microcatheter 102 (the number and/or the dimensions of the electrodes may vary as well as the spacing between sensors or electrodes); and/or (I) may be used for providing information signals to a medical-imaging system for generating (mapping) a medical image; and/or (J) be connectable to and ECG system; and/or (K) be connectable to an energy source (via one or more adapters/cables).

Referring to the embodiment as depicted in FIG. 2, the microcatheter 102 has a proximal microcatheter portion 118 and a distal microcatheter portion 120. In accordance with a preferred embodiment, the distal microcatheter portion 120 has a distal tip section supporting (configured to support) a distal energy emitter 122 (such as an electrode). The distal energy emitter 122 is configured to selectively emit energy toward the biological tissue (as previously described). The distal microcatheter portion 120 may have any shape (predefined shape, etc.).

Referring to the embodiment as depicted in FIG. 2, the microcatheter 102 is configured to be selectively connected to a medical system 124 (as needed or required). Selective connection may include (directly or indirectly) hard wired connection, wireless connection (communication), etc.

Referring to the embodiment as depicted in FIG. 2, the medical system 124 may include the energy source (described in association with FIG. 1). The energy source (also called an energy generator) is configured to provide energy (such as, radio-frequency energy, or any other form of energy) to the microcatheter 102 (once the energy source is operatively connected thereto).

Referring to the embodiment as depicted in FIG. 2, the medical system 124 may include the medical-imaging system (described in association with FIG. 1). The medical-imaging system may include a mapping system. The medical-imaging system may be utilized for assisting in the location of the microcatheter 102 and/or for identifying biological tissue (areas) to be treated, such as portions of the heart 900 of FIG. 1. The medical system 124 may include a medical-imaging system configured to generate a medical image indicating biological tissue to be treated.

Referring to the embodiment as depicted in FIG. 2, the medical system 124 may include an ECG system (known and not depicted) configured to collect (receive) an ECG signal from the microcatheter 102, for the case where the microcatheter 102 and/or the distal microcatheter portion 120 is/are configured to include at least one sensor configured to detect (and provide) an electrocardiogram signal (ECG or EKG) to the ECG system.

Referring to the embodiment as depicted in FIG. 2, the microcatheter 102 and/or the distal microcatheter portion 120 is/or configured to (preferably, in any combination and/or permutation thereof): (A) be atraumatic; and/or (B) be relatively floppy; and/or (C) be pre-shaped.

Referring to the embodiment as depicted in FIG. 2, the microcatheter 102 and/or the distal microcatheter portion 120 is/are configured to (preferably, in any combination and/or permutation thereof): (A) have an angled profile; and/or (B) have a straight profile; and/or (C) have a stiffness similar to, or greater than, a known exchange wire; and/or (D) have a fixed curve.

Referring to the embodiment as depicted in FIG. 2, the microcatheter 102 or the distal microcatheter portion 120 is configured to include at least one or more sensors (known and not depicted, such as electrodes) configured to detect the presence of biological tissue, and transmit information signals to a medical-imaging system.

Referring to the embodiment as depicted in FIG. 2, the microcatheter 102 and/or the distal microcatheter portion 120 is/are configured to include an energy emitter (known and not depicted, such as, an electrode), preferably positioned at a distal most portion of a distal microcatheter portion 120 (such as for performing trans septal puncture, etc.).

Referring to the embodiment as depicted in FIG. 2, the microcatheter 102 and/or the distal microcatheter portion 120 is/are configured to include electrical insulation (known and not depicted) to facilitate the transmission (emission) of energy.

Referring to the embodiment as depicted in FIG. 2, the microcatheter 102 and/or the distal microcatheter portion 120 is/are configured to include at least one or more visualization markers (known and not depicted).

Referring to the embodiment as depicted in FIG. 2, the microcatheter 102 and/or the distal microcatheter portion 120 is/are configured to include at least one sensor (known and not depicted) configured to detect an electrocardiogram signal (ECG or EKG).

Referring to the embodiment as depicted in FIG. 2, the microcatheter 102 may be deployed in the BAYLIS (TRADEMARK) model VERSACROSS (TRADEMARK) Transseptal wire, manufactured by the BAYLIS MEDICAL COMPANY based in Canada.

Referring to the embodiment as depicted in FIG. 2, the microcatheter 102 may be deployed in the BAYLIS (TRADEMARK) model POWER WIRE (TRADEMARK) RF guidewire, manufactured by the BAYLIS MEDICAL COMPANY based in Canada.

Referring to the embodiment as depicted in FIG. 2, the microcatheter 102 includes an electrocautery device (and any equivalent thereof, such as the BOVIE-type electrosurgical unit) configured to use electrical current to heat a metal wire that is then applied to targeted biological tissue in order to burn or coagulate the specific area of tissue (preferably, it is not used to pass the current through tissue, but rather is applied directly onto the targeted area of treatment).

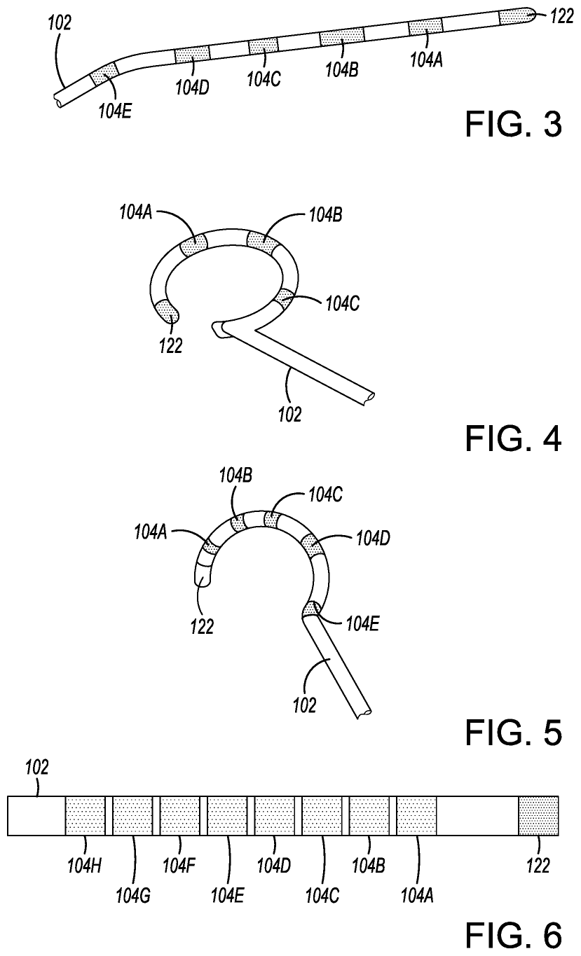

FIG. 3, FIG. 4, FIG. 5, FIG. 6 and FIG. 7 depict side views of embodiments of the microcatheter 102 of FIG. 1.

Referring to the embodiment as depicted in FIG. 3, the microcatheter 102 includes (in accordance with the first major embodiment) spaced-apart electrodes (104A, 104B, 104C, 104D, 104E) fixedly positioned along a length of the microcatheter 102.

Referring to the embodiment as depicted in FIG. 3, in general terms, the spaced-apart electrodes (104A, 104B, 104C, 104D, 104E) are configured to detect at least one (or more) signals, and transmit at least one signal (that was detected) to the medical system 124 (as depicted in FIG. 2).

Referring to the embodiment as depicted in FIG. 3, in accordance with a first option, the medical system 124 (as depicted in FIG. 2) includes the medical-imaging system (described in association with FIG. 1), and the spaced-apart electrodes (104A, 104B, 104C, 104D, 104E) are configured to detect the information signals of the biological tissue, and transmit the information signals to the medical-imaging system (for the purpose of generating a medical image of the biological tissue).

Referring to the embodiment as depicted in FIG. 3, in accordance with a second option, the medical system 124 (as depicted in FIG. 2) includes an ECG signal-processing system, and at least one electrode of the spaced-apart electrodes (104A, 104B, 104C, 104D, 104E) may be configured to detect an ECG signal, and transmit (directly or indirectly) the ECG signal to the ECG system (an ECG signal-processing system).

Referring to the embodiment as depicted in FIG. 3, the microcatheter 102 includes (in accordance with the second major embodiment) a synergistic combination of: (A) spaced-apart electrodes (104A, 104B, 104C, 104D, 104E) fixedly positioned along a length of the microcatheter 102; and (B) the distal energy emitter 122 mounted to the distal portion of the microcatheter 102. The distal most positioned one electrode of the spaced-apart electrodes (104A, 104B, 104C, 104D, 104E) is spaced apart from the distal energy emitter 122.

Referring to the embodiment as depicted in FIG. 3, the microcatheter 102 is (preferably) straight.

Referring to the embodiments as depicted in FIG. 4 and FIG. 5, the microcatheter 102 is (preferably) configured to provide any one or more of the following configurations (in any combination and/or permutation): be curved, be circular, be semi-circular, etc. (the diameter of the circle or curve may be optimum for a given medical procedure). As might be required for a specific medical procedure, the microcatheter 102 may be pre-shaped to enable optimum contact for detection and collection of an ECG signal. As might be required for a specific medical procedure, the microcatheter 102 may be pre-shaped to enable ablation of biological tissue. For instance, contact with biological tissue may be ensured by exposing the distal end of the microcatheter 102 in or for some instances in which: (A) the distal section of the microcatheter 102 is circular; and/or (B) the end of the microcatheter 102 is semicircular or banana-shaped (curved); and/or (C) the end of the microcatheter 102 is straight.

Referring to the embodiment as depicted in FIG. 6, the microcatheter 102 includes the spaced-apart electrodes (104A to 104H) fixedly positioned along a length of the microcatheter 102. The spaced-apart electrodes (104A to 104H) are, preferably, evenly spaced apart (there may be, preferably, a two (2) millimeter (mm) spacing between adjacently positioned electrodes. The distal electrode 104A and the distal energy emitter 122 may have (preferably) a five (5) millimeter spacing therebetween.

Referring to the embodiment as depicted in FIG. 7, the microcatheter 102 is positioned in the heart 900, and may be movable into the pulmonary vein 904 of the heart 900 (if so desired).

Referring to the embodiment as depicted in FIG. 7, the microcatheter 102 may be used for percutaneous access, and the transseptal puncture site may be provided through vasculature using the microcatheter 102 (such as with the Seldinger technique) for any vessel that leads to the Right Atrium (of the heart 900 as depicted in FIG. 1).

Referring to the embodiment as depicted in FIG. 7, the microcatheter 102 may be selectively activated to emit energy to the distal energy emitter 122, creating communication between the right atrium to the left atrium (RA-LA) (as depicted in FIG. 1), and the microcatheter 102 crosses into the LA; in some instances, energy may be selectively delivered only to the electrode positioned at the end of the microcatheter 102 (if desired).

Referring to the embodiment as depicted in FIG. 7, confirmation of access into the left atrium (LA) may be determined through several methods, such as: (A) fluoroscopy through the use of at least one RO (radiopaque) marker positioned on the sheath 202 and/or the microcatheter 102; and/or (B) an electro-anatomical mapping system for real-time (near real time) placement of the microcatheter 102 and the sheath 202 which may target predetermined (or located) on a CT scan (computed tomography scan), or in real-time, etc.; and/or (C) pressure differentials from the right atrium to the left atrium (RA to LA); and/or (D) injection of a contrast fluid; or (E) echogenic markers, or features on either the coil or the microcatheter 102 which may enable use of ICE or TEE for confirmation of location.

Referring to the embodiment as depicted in FIG. 7, the sheath 202 and the dilator 204 cross a biological wall, such as the fossa wall of the heart 900. The dilator 204 may then be removed leaving the sheath 202 and microcatheter 102 in the left atrium (LA). In some instances, the distal end of the microcatheter 102 may be parked in the PV (Pulmonary Vein) of the heart 900. In some instances, the sheath 202 may direct the microcatheter 102 to the PV.

Referring to the embodiment as depicted in FIG. 7, for some instances, energy may be delivered to any one electrode of the spaced-apart electrodes (104A to 104H) and/or the distal energy emitter 122 (if desired).

Referring to the embodiment as depicted in FIG. 7, for some instances, the treated region (biological tissue) may increase by slightly rotating the microcatheter 102 and re-applying energy, etc.

Referring to the embodiment as depicted in FIG. 7, for some instances, the ECG signals may be used to ensure optimal contact and/or position of any one electrode of the electrodes (104A to 104H) and the biological tissue.

Referring to the embodiment as depicted in FIG. 7, for some instances, the microcatheter 102 may be used to collect ECG signal (to confirm treatment has been completed).

Referring to the embodiment as depicted in FIG. 7, for some instances, the microcatheter 102 may be used to pace the heart 900.

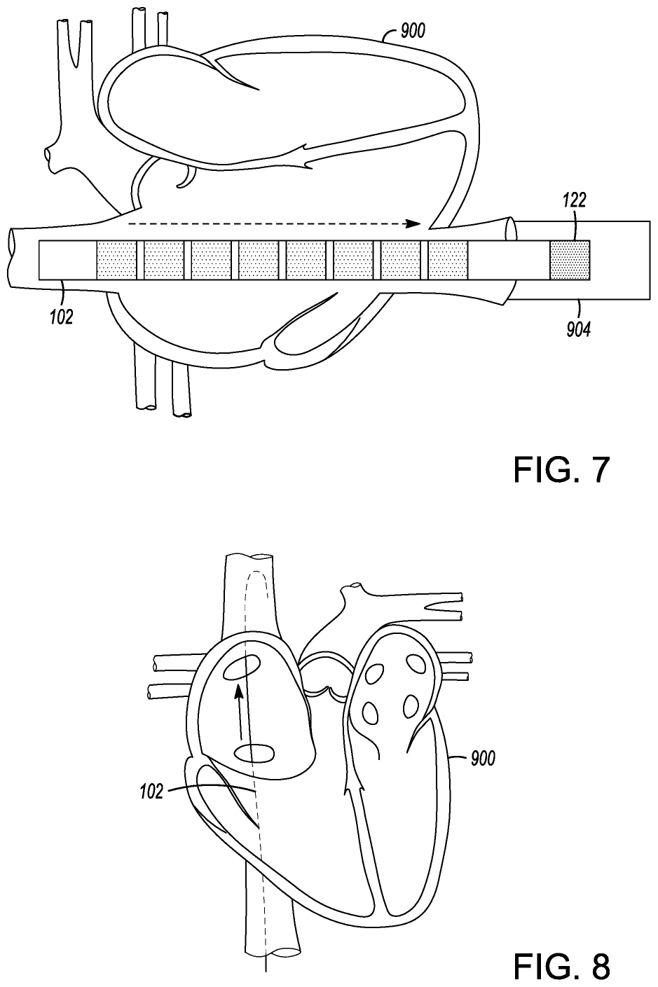

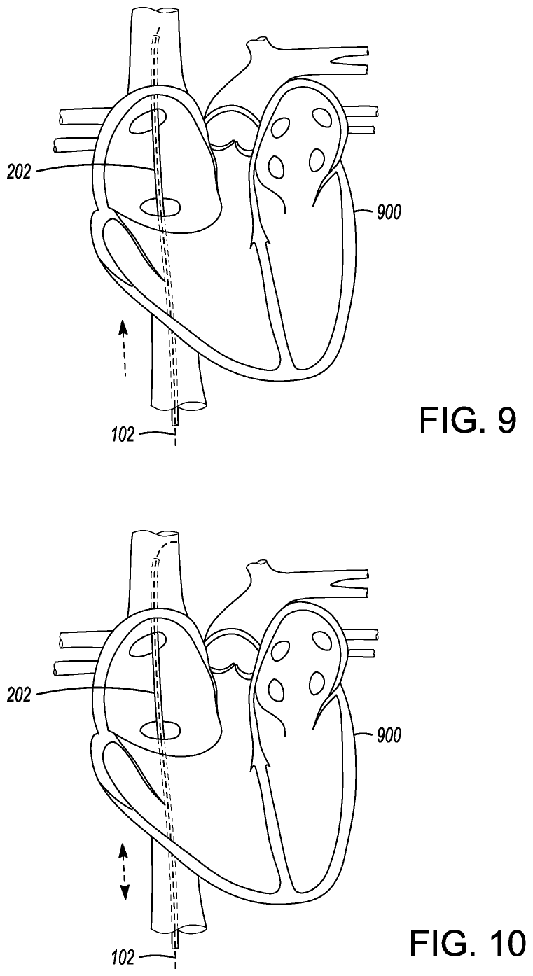

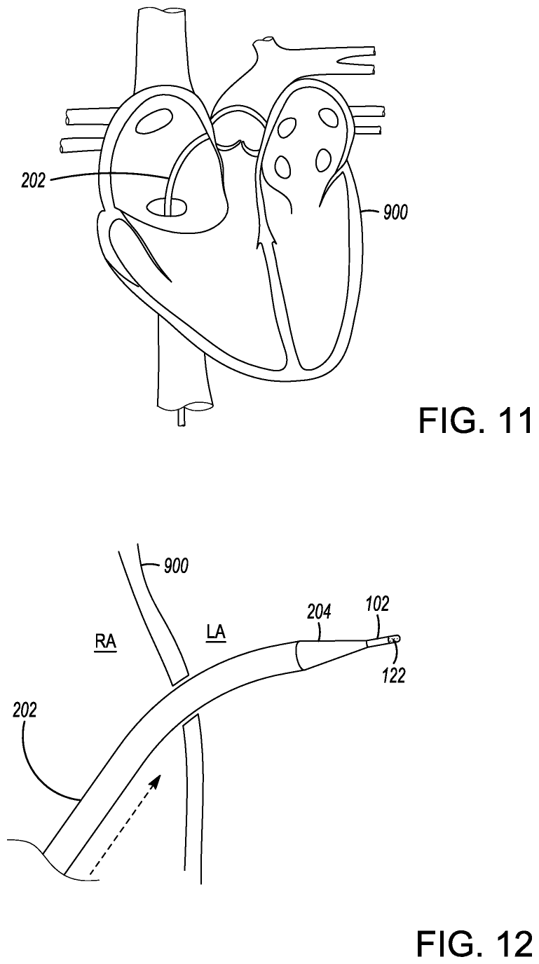

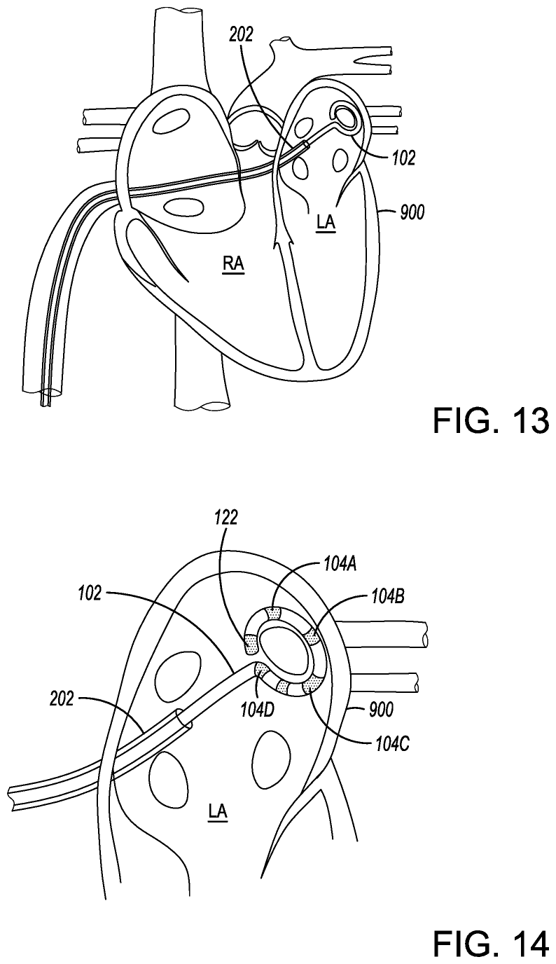

FIG. 8, FIG. 9, FIG. 10, FIG. 11, FIG. 12, FIG. 13 and FIG. 14 depict side views of embodiments of the microcatheter 102 of FIG. 1, which depict various methods (steps) for using the microcatheter 102 of FIG. 1.

Without reference to a specific FIG., a first step 501 for using the microcatheter 102 may include using the microcatheter 102 to facilitate percutaneous access (such as, femoral vein). The first step 501 may include any known (traditional) access procedure (such as, the Seldinger technique).

Referring to the embodiment as depicted in FIG. 8, a second step 502 for using the microcatheter 102 may include inserting the microcatheter 102 into the femoral vein and then to the anatomy of the heart 900, such as the right atrium (RA) of the heart 900, or the superior vena cava (SVC), etc. The microcatheter 102 may be used as a starter guidewire that may be advanced. The microcatheter 102 may be activated (used) to receive and/or detect a signal, such as ECG signals, and/or medical imaging signals for imaging mapping information (potentially without relying on fluoroscopy methods).

Referring to the embodiment as depicted in FIG. 9, a third step 503 for using the microcatheter 102 may include inserting the sheath 202 and the dilator 204 over the microcatheter 102 until the tip of the microcatheter 102 is aligned with the dilator 204.

Referring to the embodiment as depicted in FIG. 10, a fourth step 504 for using the microcatheter 102 may include: (A) establishing signal communication between the microcatheter 102 and the medical-imaging system; and (B) using the microcatheter 102 to map the RA by using a medical-imaging system (such as the EAM system). It will be appreciated that the electrodes (mounted to the microcatheter 102) are moved for the purpose of detecting biological tissue for building (forming the medical image (such as a three-dimensional map) of the tissue).

Referring to the embodiment as depicted in FIG. 11, a fifth step 505 for using the microcatheter 102 may include moving (steering) the microcatheter 102, the sheath 202 and the dilator 204 toward a biological structure, such as the fossa ovalis (during a drop-down procedure, etc.).

Referring to the embodiment as depicted in FIG. 12, a sixth step 506 for using the microcatheter 102 may include: (A) connecting the microcatheter 102 to the energy source; and (B) applying energy to the microcatheter 102 for formation of a passageway (such as, a communication passageway extending between the right atrium (RA) and the left atrium (LA), etc.). During the sixth step 506, it may be desired to: (A) disable the signal communication between the microcatheter 102 and the medical-imaging system before enabling the emission of energy from the microcatheter 102; and (B) disable connection between the microcatheter 102 and the energy generator before enabling signal communication between the microcatheter 102 and the medical-imaging system.

Without reference to a specific FIG., a seventh step 507 for using the microcatheter 102 may include moving the sheath 202 and the dilator 204 across the septum in order to enlarge the puncture site.

Without reference to a specific FIG., an eighth step 508 for using the microcatheter 102 may include using the microcatheter 102 to map the left atrium (LA) of the heart 900 by using the EAM system. This step may help in the identification of PVs (premature ventricular contractions).

Without reference to a specific FIG., a ninth step 509 for using the microcatheter 102 may include removing (retracting) the dilator 204 for special situations, such as for the case where the microcatheter 102 has a predetermined non-linear shape, and the retraction of the dilator 204 might facilitate deployment of the predetermined shape of the microcatheter 102 (to some degree). It is appreciated that the dilator 204 is used to move and control the movement of microcatheter 102 to a desired location.

Without reference to a specific FIG., a tenth step 510 for using the microcatheter 102 may include directing (moving) the microcatheter 102 toward and proximate to the portal or entrance of the PV (pulmonary vein) of the heart.

Without reference to a specific FIG., an eleventh step 511 for using the microcatheter 102 may include pushing (moving) the end section of the microcatheter 102 out of the sheath 202 so that the microcatheter 102 may assume its unstressed shape (preshaped, relaxed shape or original shape, such as a circular shape, etc.).

Referring to the embodiments as depicted in FIG. 13 and FIG. 14 (FIG. 14 is a close-up view of FIG. 13), a twelfth step 512 for using the microcatheter 102 may include placing (locating) the electrodes of the microcatheter 102 in the PV (pulmonary vein) of the heart. It will be appreciated that the portal of the PV may be mapped (by the microcatheter 102 in cooperation with the medical-imaging system). After the mapping is completed, the microcatheter 102 may be positioned and activated to treat (ablate) targeted portions of the biological tissue (for instance, the tissue may surround the portal of the PV, etc.).

Without reference to a specific FIG., a thirteenth step 513 for using the microcatheter 102 may include using the microcatheter 102 to collect the ECG signals (to identify the portions of the biological tissue that may require ablation or treatment, etc.).

Without reference to a specific FIG., a fourteenth step 514 for using the microcatheter 102 may include: (A) connecting the microcatheter 102 to the energy generator; and (B) using the microcatheter 102 to apply energy (via the electrode mounted to the microcatheter 102), for instance during a PVI procedure (for ablation of tissue, etc.). Equivalents to the energy generator may include any energy system (thermal, electrical, etc.) that may be used to ablate biological tissue.

Without reference to a specific FIG., a fifteenth step 515 for using the microcatheter 102 may include using the microcatheter 102 to collect the ECG signal (to confirm treatment completed).

Without reference to a specific FIG., a sixteenth step 516 for using the microcatheter 102 may include using the microcatheter 102 to rail other therapy devices (over the microcatheter 102) for any additional procedure.

Without reference to a specific FIG., a seventeenth step 517 for using the microcatheter 102 may include leaving the microcatheter in the heart 900, such as, in the left atrium (LA) for pacing purposes (the managed control of heart beating), for other medical therapies, etc.

Without reference to a specific FIG., an eighteenth step 518 for using the microcatheter 102 may include moving the microcatheter 102 to any other region of the heart 900 for medical treatment (diagnostic and/or pacing purposes).

Without reference to a specific FIG., a nineteenth step 519 for using the microcatheter 102 may include using the microcatheter 102 to perform PVI in a pulmonary vein in the right atrium (RA) of the heart 900.

The following is offered as further description of the embodiments, in which any one or more of any technical feature (described in the detailed description, the summary and the claims) may be combinable with any other one or more of any technical feature (described in the detailed description, the summary and the claims). It is understood that each claim in the claims section is an open ended claim unless stated otherwise. Unless otherwise specified, relational terms used in these specifications should be construed to include certain tolerances that the person skilled in the art would recognize as providing equivalent functionality. By way of example, the term perpendicular is not necessarily limited to 90.0 degrees, and may include a variation thereof that the person skilled in the art would recognize as providing equivalent functionality for the purposes described for the relevant member or element. Terms such as “about” and “substantially”, in the context of configuration, relate generally to disposition, location, or configuration that are either exact or sufficiently close to the location, disposition, or configuration of the relevant element to preserve operability of the element within the disclosure which does not materially modify the disclosure. Similarly, unless specifically made clear from its context, numerical values should be construed to include certain tolerances that the person skilled in the art would recognize as having negligible importance as they do not materially change the operability of the disclosure. It will be appreciated that the description and/or drawings identify and describe embodiments of the apparatus (either explicitly or inherently). The apparatus may include any suitable combination and/or permutation of the technical features as identified in the detailed description, as may be required and/or desired to suit a particular technical purpose and/or technical function. It will be appreciated that, where possible and suitable, any one or more of the technical features of the apparatus may be combined with any other one or more of the technical features of the apparatus (in any combination and/or permutation). It will be appreciated that persons skilled in the art would know that the technical features of each embodiment may be deployed (where possible) in other embodiments even if not expressly stated as such above. It will be appreciated that persons skilled in the art would know that other options may be possible for the configuration of the components of the apparatus to adjust to manufacturing requirements and still remain within the scope as described in at least one or more of the claims. This written description provides embodiments, including the best mode, and also enables the person skilled in the art to make and use the embodiments. The patentable scope may be defined by the claims. The written description and/or drawings may help to understand the scope of the claims. It is believed that all the crucial aspects of the disclosed subject matter have been provided in this document. It is understood, for this document, that the word “includes” is equivalent to the word “comprising” in that both words are used to signify an open-ended listing of assemblies, components, parts, etc. The term “comprising”, which is synonymous with the terms “including,” “containing,” or “characterized by,” is inclusive or open-ended and does not exclude additional, unrecited elements or method steps. Comprising (comprised of) is an “open” phrase and allows coverage of technologies that employ additional, unrecited elements. When used in a claim, the word “comprising” is the transitory verb (transitional term) that separates the preamble of the claim from the technical features of the disclosure. The foregoing has outlined the non-limiting embodiments (examples). The description is made for particular non-limiting embodiments (examples). It is understood that the non-limiting embodiments are merely illustrative as examples.

1. An apparatus usable with a medical system and biological tissue of a patient, the apparatus comprising:
a microcatheter configured to:
have a geometry being movable along a tortuous anatomy of the patient; and
be positionable, at least in part, proximate to the biological tissue; and
emit an information signal, related to the biological tissue, to the medical system so that the medical system, in use, receives the information signal from the microcatheter and processes, in use, the information signal received from the microcatheter.

2. The apparatus of claim 1, wherein:
the medical system includes a medical-imaging system; and
the microcatheter is further configured to:
detect presence of the biological tissue that is positioned or located, at least in part, proximate to the microcatheter; and
transmit the information signal, indicating detection of the presence of the biological tissue, to the medical-imaging system configured to generate a medical image, based on computations performed on the information signal that was provided by the microcatheter).

3. The apparatus of claim 1, wherein:
the microcatheter is further configured to:
be selectively connectable to an energy source; and
selectively emit, at least in part, energy toward the biological tissue for treating the biological tissue.

4. The apparatus of claim 3, wherein:
the microcatheter is configured to not emit energy while the microcatheter, in use, detects the biological tissue that is positioned proximate to the microcatheter.

5. The apparatus of claim 3, wherein:
the microcatheter is configured to not emit energy while the microcatheter, in use, transmits the information signal that is associated with the biological tissue.

6. The apparatus of claim 3, wherein:
the medical system includes a medical-imaging system; and
the microcatheter is configured to not emit energy while the microcatheter, in use, assists the surgeon in positioning the microcatheter at a desired position on a medical image formed by the medical-imaging system.

7. The apparatus of claim 3, wherein:
the microcatheter is configured to not detect biological tissue while the microcatheter, in use, selectively emits energy toward the biological tissue as indicated in a medical image that was generated from the information signal provided by the microcatheter.

8. The apparatus of claim 3, wherein:
the microcatheter is configured to:
be usable with a sheath; and
be usable with a dilator configured to be received, at least in part, into the sheath; and
be received, at least in part, into the dilator; and
wherein the sheath and the dilator are configured to be advanced over the microcatheter to a desired location.

9. The apparatus of claim 3, wherein:
the microcatheter has a proximal microcatheter portion and a distal microcatheter portion.

10. The apparatus of claim 9, wherein:
the distal microcatheter portion has a distal tip section supporting a distal energy emitter configured to selectively emit energy toward the biological tissue.

11. The apparatus of claim 3, wherein:
the microcatheter is configured to be selectively connected to the medical system.

12. The apparatus of claim 11, wherein:
the medical system includes an energy source being configured to:
be operatively connected to the microcatheter; and
provide energy to the microcatheter after the energy source is operatively connected to the microcatheter.

13. The apparatus of claim 11, wherein:
the medical system includes an ECG system configured to collect an ECG signal to be provided by the microcatheter.

14. The apparatus of claim 11, wherein:
the microcatheter is configured to include at least one sensor configured to detect an electrocardiogram signal.

15. The apparatus of claim 1, wherein:
the microcatheter includes spaced-apart electrodes fixedly positioned along a length of the microcatheter.

16. The apparatus of claim 1, wherein:
the microcatheter includes:
spaced-apart electrodes fixedly positioned along a length of the microcatheter; and
a distal energy emitter mounted to a distal portion of the microcatheter; and
a distal most positioned one electrode of the spaced-apart electrodes is spaced apart from the distal energy emitter.

17. The apparatus of claim 16, wherein:
any one electrode of the spaced-apart electrodes is configured to emit the information signal, related to the biological tissue, to the medical system; and
the distal energy emitter is configured to selectively emit, at least in part, energy toward the biological tissue for treating the biological tissue.

18. The apparatus of claim 1, wherein:
the microcatheter includes:
spaced-apart electrodes fixedly positioned along a length of the microcatheter; and
a distal energy emitter mounted to a distal portion of the microcatheter; and
a distal most positioned one electrode of the spaced-apart electrodes is spaced apart from the distal energy emitter; and
any one electrode of the spaced-apart electrodes is configured to emit the information signal, related to the biological tissue, to the medical system; and
the distal energy emitter is configured to selectively emit, at least in part, energy toward the biological tissue for treating the biological tissue; and
a selected electrode of the spaced-apart electrodes is configured to selectively emit, at least in part, energy toward the biological tissue for treating the biological tissue.

19. An apparatus, comprising:
a medical system; and
a microcatheter configured to:
be positionable, at least in part, proximate to biological tissue of a patient responsive to movement of the microcatheter along a tortuous anatomy of the patient; and
emit an information signal, related to the biological tissue, to the medical system so that the medical system, in use, receives the information signal from the microcatheter and processes, in use, the information signal received from the microcatheter.

20. A method usable with a medical system and biological tissue of a patient, the method comprising:
using a microcatheter configured to:
have a geometry being movable along a tortuous anatomy of the patient; and
be positionable, at least in part, proximate to the biological tissue; and
emit an information signal, related to the biological tissue, to the medical system so that the medical system, in use, receives the information signal from the microcatheter and processes, in use, the information signal received from the microcatheter.
